(19) United States
(12) Patent Application Publication
(10) Pub. No.: US 2009/0163551 A1
(43) Pub. Date: Jun. 25, 2009

(54) Compositions and Methods for Treating Diseases

(75) Inventor: David Lewis Earnest, Morristown, NJ (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: Novartis AG, Basel (CH)

(21) Appl. No.: 12/308,163

(22) PCT Filed: Jun. 13, 2007
(86) PCT No.: PCT/US2007/071080

§ 371 Date: Dec. 8, 2008

Related U.S. Application Data
(60) Provisional application No. 60/813,852, filed on Jun. 15, 2006.

(51) Int. Cl. A61K 31/4439; A61K 31/404

(52) U.S. Cl. 514/338; 514/415

(57) ABSTRACT

This invention relates to methods for treating, preventing and/or managing gastric injury, e.g., NSAID-induced gastric injury in a subject including administering to the subject tegaserod, alone or in combination with a proton pump inhibitor. Also provided are compositions and kits for use in methods of the invention.

### RELATED APPLICATION

This application claims priority to U.S. Provisional Application No. 60/813,852, filed Jun. 15, 2006. The contents of any patents, patent applications, and references cited throughout this specification are hereby incorporated by reference in their entireties.

### FIELD OF THE INVENTION

This invention relates to a method for preventing gastric injury, e.g., mucosal injury and gastric muscle dysfunction, by administering tegaserod, alone or in combination with a proton pump inhibitor, to a subject in need thereof.

### BACKGROUND OF THE INVENTION

Non-steroidal anti-inflammatory drugs (NSAIDs) are the most frequently prescribed drugs for the treatment of pain associated with a variety of musculoskeletal and inflammatory disorders. In the United States, about 100 million prescriptions are written each year to provide effective relief of pain and treatment of inflammatory diseases. Commonly used NSAIDs include sulindac, naproxen, indomethacin, mefenamic acid, diclofenac, fenoprofen, and diflunisal.

However, considerable evidence indicates that NSAIDs have frequent, serious, and costly gastrointestinal tract toxic side effects. These include mild dyspepsia, gastritis, peptic ulcer disease, as well as more serious gastrointestinal complications such as bleeding and perforation, leading sometimes to significant morbidity and, to a lesser extent, mortality. Serious GI complications due to NSAID use represent the greatest threat to life in patients with connective tissue diseases, second only to the primary disease and its complications.

Other commonly encountered gastrointestinal disorders include inflammatory bowel disorders (IBD) and functional bowel disorders (FBD), including dyspepsia. These GI disorders include a wide range of disease states that are currently only moderately controlled, including Crohn's disease, ileitis, ischemic bowel disease, and ulcerative colitis, as well as IBD, the irritable bowel syndrome, dyspepsia, and gastro-esophageal reflux for FBD, and other forms of visceral pain.

There is, therefore, a great need for the development of compositions for use in the treatment of gastric injury, including gastric injury caused by NSAD use.

### SUMMARY OF THE INVENTION

There remains a need for new treatments and therapies for gastric injury, including mucosal injury and gastric muscle dysfunction. There is also a need for compositions useful in the treatment, prevention or amelioration of one or more symptoms of gastric injury. Furthermore, there is a need for new treatments and therapies for gastric injury caused by NSAID use.

In one aspect, the invention provides a pharmaceutical composition comprising tegaserod and a proton pump inhibitor. In one embodiment, the proton pump inhibitor is selected from the group consisting of omeprazole, lansoprazole, rabeprazole, pantoprazole and leminoprazole. In another embodiment, the proton pump inhibitor is omeprazole.

In another aspect, the invention provides a method of treating or preventing gastric injury in a subject in need thereof, comprising administering to the subject an effective amount of tegaserod. In one embodiment, the gastric injury is associated with NSAID administration. In another embodiment, the tegaserod is administered in combination with a proton pump inhibitor, wherein the proton pump inhibitor is omeprazole, lansoprazole, rabeprazole, pantoprazole or leminoprazole. In a particular embodiment, the proton pump inhibitor is omeprazole. In one embodiment of the treatment, tegaserod is administered first followed by administration of a proton pump inhibitor. In another embodiment of the treatment, the proton pump inhibitor is administered first followed by administration of tegaserod. In still another embodiment of the treatment, tegaserod and the proton pump inhibitor are administered simultaneously.

In one embodiment, the gastric injury that will be treated using the methods of the invention is selected from the group consisting of mucosal injury, gastric muscle dysfunction, gastritis, peptic erosions, ulcerations and gastric lesions.

In another aspect, the invention provides a method of treating pain and/or inflammation in a subject, said method comprising administering to the subject in need thereof a therapeutically-effective amount of a pharmaceutical composition comprising an NSAID and tegaserod.

In one embodiment, the NSAID is selected from the group consisting of salicylic acid, aspirin, methyl salicylate, diflunisal, salsalate, olsalazine, sulfasalazine, acetaminophen, sulindac, etodolac, tolmetin, ketorolac, diclofenac, ibuprofen, naproxen, fenoprofen, ketoprofen, flurbiprofen, oxaprozin, indomethacin, piroxicam, celecoxib and rofecoxib.

In another embodiment, the combination of an NSIAD and tegaserod may be used to treat inflammation in a subject, wherein the inflammation is selected from the group consisting of fever, arthritis, asthma, bronchitis, menstrual cramps, tendinitis, bursitis, inflammatory disorders of the skin, gastrointestinal conditions, vascular diseases, migraine headaches, periarteritis nodosa, thyroidiris, aplastic anemia, Hodgkin's disease, sclerodoma, rheumatic fever, myasthenia gravis, sarcoidosis, nephrotic syndrome, irritable bowel syndrome, functional dyspepsia, Behcet's syndrome, polymyositis, hypersensitivity, conjunctivitis, gingivitis, swelling occurring after injury and myocardial ischemia. In one embodiment, the arthritis is selected from rheumatoid arthritis, spondyloarthopathies, gouty arthritis, systemic lupus erythematosus, osteoarthritis and juvenile arthritis.

In another embodiment, the combination of an NSIAD and tegaserod may be used to treat pain in a subject, wherein the pain is selected from the group consisting of menstrual pain, low back pain, neck pain, skeletal pain, post-partum pain, headache, pain associated with migraine, toothache, sprains, strains, arthritis, degenerative joint diseases, gout, ankylosing spondylitis, bursitis, burns, including radiation and corrosive chemical injuries, sunburns, bone fracture, immune and autoimmune diseases, cellular neoplastic transformations or metastic tumor growth, and pain following surgical and dental procedures.

In another aspect, the invention provides a method of treating mucosal injury and/or gastric muscle dysfunction in a subject in need thereof, comprising administering to the subject an effective amount of tegaserod. In one embodiment, the tegaserod is administered in combination with omeprazole. In another embodiment, the subject is human.

### BRIEF DESCRIPTION OF THE DRAWINGS

FIG. 1 demonstrates the effect of pretreatment with tegaserod (1 mg/kg), omeprazole (20 mg/kg) or a combination of both drugs in a rat model of indomethacin-induced gastric mucosal ulceration (1A) and myeloperoxidase activity (1B). Drugs were administered b.i.d. one day before the experiment and 30 minutes prior to indomethacin on the day of experiment. Gastric mucosal injury was evaluated 6 h after intragastric instillation of 50 mg/kg indomethacin. Data are mean±S.E.M. from 6 rats in each group. Statistical significance of differences between groups was assessed by one-way ANOVA followed by Bonferroni multiple comparison test. ** p<001,*** p<0.001 compared to the vehicle-treated group.

FIG. 2 demonstrates the effect of pretreatment of the rats with tegaserod (1 mg/kg), omeprazole (20 mg/kg) or a combination of both drugs on the contractile response to KCl (80 mM) in circular muscle strips from the gastric antrum of rats with indomethacin-induced gastric damage. Data are mean±S.E.M. from 6 rats in each group. Statistical significance of differences between groups was assessed by one-way ANOVA followed by Bonferroni multiple comparison test. ** p<0.01 compared to the vehicle-treated group.

FIG. 3 demonstrates the effect of pretreatment of the rats with tegaserod (1 mg/kg), omeprazole (20 mg/kg) or a combination of both drugs on the contractile responses to carbachol in circular muscle strips from the gastric antrum of rats with indomethacin-induced gastric damage. FIG. 3A shows concentration-response curves obtained by cumulative addition of carbachol (1 nM-1 μM) to the bathing solution. FIG. 3B shows maximal responses to carbachol. Data are mean±S.E.M. from 6 rats in each group. Statistical significance of differences between groups was assessed by Kruskal-Willis non-parametric ANOVA followed by Dunn's multiple comparison test. *** p<0.001 compared to vehicle-treated group.

FIG. 4 demonstrates the effect of pretreatment of the rats with tegaserod (1 mg/kg), omeprazole (20 mg/kg) or a combination of both drugs on the contractile responses to 5-HT in circular muscle strips from the gastric antrum of rats with indomethacin-induced gastric damage. FIG. 4A shows concentration-response curves achieved by cumulative addition of 5-HT (1 nM-10 μM) to the bathing solution. FIG. 4B shows maximal responses to 5-HT. Data are mean±S.E.M. from 6 rats in each group. Statistical significance of differences between groups was assessed by one-way ANOVA followed by Bonferroni multiple comparison test. * p<0.05, ** p<0.01 compared to vehicle-treated group.

FIG. 5 demonstrates neurally mediated responses induced by EFS (0.5 ms, 1-15 Hz) in circular muscle strips isolated from the gastric antrum of naïve rats or rats with indomethacin-induced gastric damage. Data are mean±S.E.M. from 6 rats in each group. Differences were assessed by one-way ANOVA followed by Bonferroni multiple comparison test. * p<0.05 compared to responses in naïve rats induced by the same stimulus frequency.

FIG. 6 shows regression lines illustrating the frequency-dependent contractile responses to EFS (0.5 ms. 1-15 Hz) in circular muscle strips from the gastric antrum. Pretreatment of the rats with tegaserod (1 mg/kg), omeprazole (20 mg/kg) or a combination of both drugs protected the linear increase in the amplitude of contractile responses with the increase in EFS frequency (slopes are presented in Table 1). Regression analysis performed on data from 6 rats in each group.

### DETAILED DESCRIPTION OF THE INVENTION

The present invention features methods of treating, preventing and/or managing gastric injury and disorders associated therewith, and compositions for use in such methods. It is understood that compositions and methods described herein may either be used to treat, prevent or manage a disorder associated with gastric injury or for treating and/or preventing the gastric injury itself. In some instances, the gastric injury is a component of a disorder. In other instances, the gastric injury may either be a cause or a symptom of a disorder. In still other instances, the gastric injury is caused by NSAID use.

In particular, this invention is directed to compositions, e.g., a combination of tegaserod and PPI, as well as pharmaceutical compositions containing the tegaserod/PPI combination, for use in treatment of gastric injury, e.g., mucosal injury and gastric muscle dysfunction.

In one aspect, the invention is directed toward use of tegaserod for treatment of gastric injury in a subject. A pharmaceutically acceptable salt of tegaserod is also useful for the purposes of this invention. In one embodiment, the gastric injury is associated with NSAID administration. In another embodiment, the gastric injury is a mucosal injury and gastric muscle dysfunction.

Tegaserod is chemically known as 3-(5-methoxy-1H-indol-3-ylmethylene)-N-pentylcarbazimidamide. Its preparation is disclosed in U.S. Pat. No. 5,510,353, as well as U.S. patent application Ser. No. 11/315,859. Both of these documents are incorporated herein by reference in their entirety.

Proton pump inhibitors (PPI) are potent inhibitors of gastric acid secretion, inhibiting H⁺, K⁺-ATPase, the enzyme involved in the final step of hydrogen ion production in the parietal cells. The term proton pump inhibitor includes, but is not limited to, omeprazole, lansoprazole, rabeprazole, pantoprazole and leminoprazole, including isomers, enantiomers and tautomers thereof and alkaline salts thereof. Proton pump inhibitors typically include benzimidazole compounds. The following patents describe various benzimidazole compounds suitable for use in the invention described herein: U.S. Pat. No. 4,045,563, U.S. Pat. No. 4,255,431, U.S. Pat. No. 4,359,465, U.S. Pat. No. 4,472,409, U.S. Pat. No. 4,508,905, JP-A-59181277, U.S. Pat. No. 4,628,098, U.S. Pat. No. 4,738,975, U.S. Pat. No. 5,045,321, U.S. Pat. No. 4,786,505, U.S. Pat. No. 4,853,230, U.S. Pat. No. 5,045,552, EP-A-295603, U.S. Pat. No. 5,312,824 and GB 2,163,747. All of the above patents are hereby incorporated by reference. Proton pump inhibitors, e.g., omeprazole and its pharmaceutically acceptable salts, which are used in accordance with the invention are known compounds and can be produced by known processes. In certain preferred embodiments, the proton pump inhibitor is omeprazole, either in racemic mixture or only the (−) enantiomer of omeprazole (i.e., esomeprazole), as set forth in U.S. Pat. No. 5,877,192, hereby incorporated by reference.

In particular embodiments, the PPI to be used in combination with tegaserod is omeprazole.

In certain embodiments, the compositions of the present invention (e.g., tegaserod, or tegaserod in combination with a PPI, or tegaserod in combination with and NSAID) is further characterized as a modulator of gastric injury, including, but not limited to, mucosal injury and gastric muscle dysfunction.

In other embodiments, the compositions of the present invention are used for the treatment of gastric injury a subject. In certain embodiments, gastric injury includes include inflammatory bowel disorders (IBD) and functional bowel disorders (FBD), including dyspepsia; Crohn's disease, ileitis, ischemic bowel disease, and ulcerative colitis, dyspepsia, and gastro-esophageal reflux for FBD, and forms of visceral pain. In certain embodiments, the gastric injury is induced by the use of an NSAID by a subject. In particular embodiments, the gastric injury is a mucosal injury, gastric muscle dysfunction, gastritis, a peptic erosion, ulceration or gastric lesion.

In certain embodiments, the invention provides a pharmaceutical composition of any of the compositions of the present invention. In a related embodiment, the invention provides a pharmaceutical composition of any of the compositions of the present invention and a pharmaceutically acceptable carrier or excipient of any of these compositions. In certain embodiments, the invention includes a pharmaceutical composition comprising tegaserod, tegaserod and omeprazole, and tegaserod and an NSAID.

The compositions of the present invention are suitable as active agents in pharmaceutical compositions that are efficacious particularly for treating gastric injury, e.g., NSAID-induced gastric injury. The pharmaceutical composition in various embodiments has a pharmaceutically effective amount of the present active agent along with other pharmaceutically acceptable excipients, carriers, fillers, diluents and the like. The phrase, “pharmaceutically effective amount” as used herein indicates an amount necessary to administer to a host, or to a cell, issue, or organ of a host, to achieve a therapeutic result, especially the regulating, modulating or inhibiting gastric injury, e.g., NSAID-induced gastric injury.

In other embodiments, the present invention provides a use of any of the compositions of the invention for manufacture of a medicament to treat gastric injury in a subject. In other embodiments, the invention provides a method of manufacture of a medicament, including formulating any of the compositions of the present invention for treatment of a subject.

In some embodiments, a method of treating gastric injury in a subject includes administering to a subject in need thereof an effective amount of tegaserod. In other embodiments, a method of treating gastric injury in a subject includes administering to a subject a first amount of tegaserod and at least one second PPI, e.g., omeprazole. Such a treatment can either be additive or synergistic, compared to the effect on gastric injury in the presence of tegaserod or the PPI when given alone. In some embodiments, administration of tegaserod and a PPI results in a synergistic increase in gastric injury treatment. Furthermore, normal amounts of each compound when given in combination could provide for greater efficacy in subjects who are either unresponsive or minimally responsive to each compound when used alone.

In a particular embodiment, methods featured herein include administering a combination of tegaserod and omeprazole for treating gastric injury, such as, for example, mucosal injury and gastric muscle dysfunction.

In some embodiments, a first dose of tegaserod and at least one second dose of a PPI are included in a single composition, which is administered to a subject with a gastric injury. In other embodiments, a first dose of tegaserod and at least one second dose of a PPI are administered separately to such a subject. The first and at least one second dose may either be co-administered to a subject (i.e., at the same time) or be administered sequentially (i.e., one after the other).

In another embodiment, tegaserod can be administered to a subject in need thereof in combination with an NSAID for the treatment of pain and/or inflammation. The term “NSAID”, as used herein, represents a nonsteroidal anti-inflammatory agent which can be identified as such by the skilled artisan. NSAIDs are known for their inhibition of cyclooxygenases I and II, the enzymes responsible for the biosynthesis of the prostaglandins and certain related autacoids. NSAIDs are known to be antipyretic, analgesic, and antiinflammatory. The term NSAID shall, in addition, refer to any compound acting as a non-steriodal antiinflammatory agent. For example, The Pharmacological Basis of Therapeutics, 9th edition, Macmillan Publishing Co., 1996, pp 617-655, provides well-known examples of NSAIDs. The term includes, but is not limited to, salicylic acid derivatives, such as salicylic acid, aspirin, methyl salicylate, diflunisal, salsalate, olsalazine and sulfasalazine; para-aminophenol derivatives, such as acetaminophen; sulindac; etodolac; tolmetin; ketorolac; diclofenac; propionic acid derivatives, such as ibuprofen, naproxen, fenoprofen, ketoprofen, flurbiprofen and oxaprozin; acetic acid derivatives, such as indomethacin; enolic acids, such as piroxicam; and cyclooxygenase II inhibitors, such as celecoxib, lumiracoxib and rofecoxib. Applicants appreciate that new NSAIDs may be in development, and the present invention contemplates a synergistic combination and compositions comprising such new agents with duloxetine as well.

Preferably, the NSAID is selected from aspirin, ibuprofen, naproxen, celecoxib and rofecoxib.

Compositions described herein can be prepared, e.g., by combining effective amounts of a first dose and a second dose, with a pharmaceutically acceptable carrier, which may take a wide variety of forms depending on the form of preparation desired for administration. An example of a pharmaceutically acceptable carrier used in the compositions described herein is DMSO, e.g., 0.1% DMSO.

In some embodiments, pharmaceutical compositions can be in unitary dosage form suitable for administration orally, rectally or by parenteral injection. For example, in preparing compositions in oral dosage form, any of the usual pharmaceutical media may be employed, such as, e.g., water, glycols, oils, alcohols and the like, as in the case of oral liquid preparations, such as suspensions, syrups, elixirs and solutions; or solid carriers, such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are employed. For parenteral compositions, carriers usually comprise sterile water, at least in large part, though other ingredients, e.g., to aid solubility, may be included. Injectable solutions, e.g., are prepared using a carrier which comprises saline solution, glucose solution or a mixture of saline and glucose solution. Injectable suspensions may also be prepared in which case appropriate liquid carriers, suspending agents and the like may be employed. In case of compositions suitable for percutaneous administration, carrier optionally comprises a penetration enhancing agent and/or a suitable wetting agent, which may be combined with suitable additives of any nature in minor proportions, which additives do not cause a significant deleterious effect to the skin. Additives may facilitate the administration to the skin and/or may be helpful for preparing desired compositions. These compositions may be administered in various ways, e.g., as a transdermal patch, as a spot-on, as an ointment.

It is especially advantageous to formulate the pharmaceutical compositions described herein in dosage unit form for ease of administration and uniformity of dosage. Dosage unit form, as used herein, refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

In general it is contemplated that a therapeutically effective amount of a first or a second dose would be from about 0.0001 mg/Kg to 0.001 mg/Kg; 0.001 mg/kg to about 10 mg/kg body weight or from about 0.02 mg/kg to about 5 mg/kg body weight. In some embodiments, a therapeutically effective amount of a first or a second dose is from about 0.007 mg to about 0.07 mg, about 0.07 mg to about 700 mg, or from about 1.4 mg to about 350 mg. A method of prophylactic or curative treatment may also include administering the composition in a regimen of between one to five intakes per day.

In some embodiments, a therapeutically effective amount of a first dose or a second dose includes, but is not limited to, the amount less than about 0.01 mg/dose, or less than about 0.5 mg/dose, or less than about 1 mg/dose, or less than about 2 mg/dose, or less than about 5 mg/dose, or less than about 10 mg/dose, or less than about 20 mg/dose, or less than about 25 mg/dose, or less than about 50 mg/dose, or less than about 100 mg/dose. The number of times a day a first or a second dose is administrated to a subject can be determined based on various criteria commonly used in the art and/or those described herein.

A combination of compounds described herein can either result in synergistic increase in gastric injury treatment, or such an increase can be additive. Compositions described herein can include lower dosages of each compound in a composition, thereby avoiding adverse interactions between compounds and/or harmful side effects, such as ones which have been reported for similar compounds. Furthermore, normal amounts of each compound when given in combination could provide for greater efficacy in subjects who are either unresponsive or minimally responsive to each compound when used alone.

A synergistic effect can be calculated, e.g., using suitable methods, such as the Sigmoid-Emax equation (Holford, N. H. G. and Scheiner, L. B., Clin. Pharmacokinet. 6: 429-453 (1981)), the equation of Loewe additivity (Loewe, S, and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

As discussed above, an advantage of the compositions described herein is the ability to use less of each compound than is needed when each is administered alone. Another advantage is that greater efficacy may be achieved in subjects who are either unresponsive or minimally responsive to each compound when used alone in normal amounts by giving the agents in combination. As such, undesirable side effects associated with the compounds are reduced (partially or completely) and/or improved efficacy may be achieved. A reduction in side effects with or without improved efficacy can result in increased patient compliance over current treatments.

### DEFINITIONS

The term “treat”, “treated”, “treating” or “treatment” includes the diminishment or alleviation of at least one symptom associated or caused by the state, disorder or disease being treated. In certain embodiments, the treatment comprises the induction of a gastric injury, followed by the activation of the composition of the invention, which would in turn diminish or alleviate at least one symptom associated or caused by the gastric injury being treated. For example, treatment can be diminishment of one or several symptoms of a disorder or complete eradication of a disorder.

The term “use” includes any one or more of the following embodiments of the invention, respectively: the use in the treatment of a gastric injury; the use for the manufacture of pharmaceutical compositions for use in the treatment of these diseases, e.g., in the manufacture of a medicament; methods of use of compositions of the invention in the treatment of these diseases; pharmaceutical preparations having compositions of the invention for the treatment of these diseases; and compositions of the invention for use in the treatment of these diseases; as appropriate and expedient, if not stated otherwise. In particular, diseases to be treated and are thus preferred for use of a composition of the present invention are gastric injuries, e.g., mucosal injuries and gastric muscle dysfunction.

The term “subject” is intended to include organisms, e.g., prokaryotes and eukaryotes, which are capable of suffering from or afflicted with a gastric injury. Examples of subjects include mammals, e.g., humans, dogs, cows, horses, pigs, sheep, goats, cats, mice, rabbits, rats and transgenic non-human animals. In certain embodiments, the subject is a human, e.g., a human suffering from, at risk of suffering from, or potentially capable of suffering from a gastric injury. In another embodiment, the subject is a cell.

As used herein, “therapeutically effective amount” refers to an amount of a first dose and a second dose, as used herein, sufficient to elicit a desired biological response. In case of the methods described herein, a desired biological response is a reduction (complete or partial) of at least one symptom associated with the disorder being treated and/or improved efficacy. As with any treatment, particularly treatment of a multi-symptom disorder, it is advantageous to treat as many disorder-related symptoms as the patient experiences. The phrase “therapeutically effective amount” encompasses amounts of a dose of tegaserod, as well as amounts of a first dose of tegaserod and a dose of a PPI, as well as amounts of a first dose of tegaserod and a dose of an NSAID, as described herein, wherein the combination of the first and at least one second dose results in gastric injury treatment. Any amounts of a first dose and a second dose as described herein can be used in the prevention, treatment, and/or management of a disorder, as described herein.

As used herein, the term “pharmaceutically acceptable excipient” includes compounds that are compatible with the other ingredients in a pharmaceutical formulation and not injurious to the subject when administered in therapeutically effective amounts.

As used herein, the term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric and phosphoric acid. Organic acids may be aliphatic, aromatic, carboxylic and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic and the like.

### Co-Administration

In certain embodiments, co-administration of a first dose of tegaserod and a second amount of a PPI results in an increased therapeutic effect, or a first dose of tegaserod and a second amount of an NSAID, relative to the effect resulting from separate administration of the first dosage amount or the second dosage amount. In some embodiments, an increased therapeutic effect is an additive effect. In some other embodiments, an increased therapeutic effect is a synergistic effect. In certain embodiments, the amounts of each of the first and second doses are less than the amounts used when each compound is administered alone, thereby lessening or alleviating at least one detrimental side effect associated with single administration of either of the compounds.

Compositions used in methods described herein may either be co-administered or be administered sequentially. As used herein, “co-administration” of a first and at least one second compound refers to the simultaneous delivery of two or more separate chemical entities (e.g., tegaserod and a PPI, e.g., omeprazole), whether in vitro or in vivo (e.g., to a subject). In some embodiments, compounds that are coadministered work in conjunction with each other (e.g., to treat gastric injury). Co-administration encompasses administration of the first and second amounts of the compounds in an essentially simultaneous manner such as, for example, in a single pharmaceutical composition, e.g., capsule or tablet having a fixed ratio of first and second amounts, or in multiple, separate capsules or tablets for each. “Sequential administration”, refers to separate administration of each compound in a sequential manner in either order. When administration involves the separate administration (e.g., sequential administration) of the first compound and a second compound, as described herein, the compounds are administered sufficiently close in time to have the desired therapeutic effect. For example, the period of time between each administration, which can result in the desired therapeutic effect, can range from minutes to hours and can be determined based on the properties of each compound such as potency, solubility, bioavailability, plasma half-life and kinetic profile. For example, the compounds can be administered in any order within about 24 hours of each other or within any time less than 24 hours of each other.

A suitable dose per day for tegaserod can be in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

A suitable dose per day for combination of the compounds, i.e., a first dose of tegaserod, and a second dose of a PPI or an NSAID, can be in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

### Pharmaceutical Compositions

The language “effective amount” of the composition is that amount necessary or sufficient to treat or prevent a gastric injury, e.g., prevent the various morphological and somatic symptoms of a gastric injury, and/or a disease or condition described herein. In an example, an effective amount of the composition of the invention is the amount sufficient to treat a gastric injury in a subject. The effective amount can vary depending on such factors as the size and weight of the subject, the type of illness, or the particular composition of the invention. For example, the choice of the composition of the invention can affect what constitutes an “effective amount”. One of ordinary skill in the art would be able to study the factors contained herein and make the determination regarding the effective amount of the compositions of the invention without undue experimentation.

The regimen of administration can affect what constitutes an effective amount. The composition of the invention can be administered to the subject either prior to or after the onset of a gastric injury. Further, several divided dosages, as well as staggered dosages, can be administered daily or sequentially, or the dose can be continuously infused, or can be a bolus injection. Further, the dosages of the composition(s) of the invention can be proportionally increased or decreased as indicated by the exigencies of the therapeutic or prophylactic situation.

Compositions of the invention (e.g., tegaserod, or a composition comprising a dose of tegaserod and a PPI) may be used in the treatment of states, disorders or diseases as described herein, or for the manufacture of pharmaceutical compositions for use in the treatment of these diseases. Methods of use of compositions of the present invention in the treatment of these diseases, or pharmaceutical preparations having compositions of the present invention for the treatment of these diseases.

The language “pharmaceutical composition” includes preparations suitable for administration to mammals, e.g., humans. When the compositions of the present invention are administered as pharmaceuticals to mammals, e.g., humans, they can be given per se or as a pharmaceutical composition containing, e.g., 0.1-99.5% (more preferably, 0.5-90%) of active ingredient in combination with a pharmaceutically acceptable carrier.

The phrase “pharmaceutically acceptable carrier” is art recognized and includes a pharmaceutically acceptable material, composition or vehicle, suitable for administering compositions of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid and the like.

Formulations of the present invention include those suitable for oral, nasal, topical, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the composition that produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a composition of the present invention with the carrier and, optionally, one or more accessory ingredients. In general, the formulations are prepared by uniformly and intimately bringing into association a composition of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a composition of the present invention as an active ingredient. A composition of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, e.g., carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, e.g., cetyl alcohol and glycerol monostearate; absorbents, such as kaolin and bentonite clay; lubricants, such a talc, calcium stearate, magnesium stearate, solid polyethylene glycols, sodium lauryl sulfate and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (e.g., sodium starch glycolate or cross-linked sodium carboxymethyl cellulose), surface-active or dispersing agent. Molded tablets may be made by molding in a suitable machine a mixture of the powdered composition moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, e.g., hydroxypropylmethyl cellulose in varying proportions to provide the desired release profile, other polymer matrices, liposomes and/or microspheres. They may be sterilized by, e.g., filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compositions of the invention include pharmaceutically acceptable emulsions, microemulsions, solutions, suspensions, syrups and elixirs. In addition to the active ingredient, the liquid dosage forms may contain inert diluent commonly used in the art, such as, e.g., water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

Besides inert diluents, the oral compositions can also include adjuvants, such as wetting agents, emulsifying and suspending agents, sweetening, flavoring, coloring, perfuming and preservative agents.

Suspensions, in addition to the active compositions, may contain suspending agents as, e.g., ethoxylated isostearyl alcohols, polyoxyethylene sorbitol and sorbitan esters, microcrystalline cellulose, aluminum metahydroxide, bentonite, agar-agar and tragacanth and mixtures thereof.

Formulations of the pharmaceutical compositions of the invention for rectal or vaginal administration may be presented as a suppository, which may be prepared by mixing one or more compositions of the invention with one or more suitable nonirritating excipients or carriers comprising, e.g., cocoa butter, polyethylene glycol, a suppository wax or a salicylate, and which is solid at room temperature, but liquid at body temperature and, therefore, will melt in the rectum or vaginal cavity and release the active composition.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a composition of this invention include powders, sprays, ointments, pastes, creams, lotions, gels, solutions, patches and inhalants. The active composition may be mixed under sterile conditions with a pharmaceutically acceptable carrier, and with any preservatives, buffers or propellants that may be required.

The ointments, pastes, creams and gels may contain, in addition to an active composition of this invention, excipients, such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to a composition of this invention, excipients, such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a composition of the present invention to the body. Such dosage forms can be made by dissolving or dispersing the composition in the proper medium. Absorption enhancers can also be used to increase the flux of the composition across the skin. The rate of such flux can be controlled by either providing a rate controlling membrane or dispersing the active composition in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compositions of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol and the like), and suitable mixtures thereof, vegetable oils, such as olive oil, and injectable organic esters, such as ethyl oleate. Proper fluidity can be maintained, e.g., by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, e.g., paraben, chlorobutanol, phenol sorbic acid and the like. It may also be desirable to include isotonic agents, such as sugars, sodium chloride, and the like into the compositions. In addition, prolonged absorption of the injectable pharmaceutical form may be brought about by the inclusion of agents that delay absorption, such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral and/or IV administration is preferred.

The phrases “parenteral administration” and “administered parenterally”, as used herein, means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration”, “administered systemically”, “peripheral administration” and “administered peripherally”, as used herein, mean the administration of a compound, drug or other material other than directly into the central nervous system, such that it enters the patient's system and, thus, is subject to metabolism and other like processes, e.g., subcutaneous administration.

These compounds may be administered to humans and other animals for therapy by any suitable route of administration, including orally, nasally, as by, e.g., a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well-known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats gastric injury.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

### Kits

The present invention also provides kits for use by a consumer for treating disease. The kits comprise:
a) a pharmaceutical composition comprising a compound of the invention (e.g., tegaserod, or tegaserod in combination with a PPI, e.g., omeprazole) and a pharmaceutically acceptable carrier, vehicle or diluent; and, optionally,
b) instructions describing a method of using the pharmaceutical composition for treating the specific disease.
   The instructions may also indicate that the kit is for treating disease while substantially reducing the concomitant liability of adverse effects associated with estrogen administration.

A “kit” as used in the instant application includes a container for containing the separate unit dosage forms such as a divided bottle or a divided foil packet. The container can be in any conventional shape or form as known in the art which is made of a pharmaceutically acceptable material, e.g., a paper or cardboard box, a glass or plastic bottle or jar, a re-sealable bag (e.g., to hold a “refill” of tablets for placement into a different container), or a blister pack with individual doses for pressing out of the pack according to a therapeutic schedule. The container employed can depend on the exact dosage form involved, e.g. a conventional cardboard box would not generally be used to hold a liquid suspension. It is feasible that more than one container can be used together in a single package to market a single dosage form. For example, tablets may be contained in a bottle which is in turn contained within a box.

An example of such a kit is a so-called blister pack. Blister packs are well-known in the packaging industry and are being widely used for the packaging of pharmaceutical unit dosage forms (tablets, capsules and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card, e.g., as follows “First Week, Monday, Tuesday”, . . . etc. . . . “Second Week, Monday, Tuesday . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that, has been dispensed. Another example of such a memory-aid is a battery-powered micro-chip memory coupled with a liquid crystal readout, or audible reminder signal which, e.g., reads out the date that the last daily dose has been taken and/or reminds one when the next dose is to be taken.

### Exemplification of the Invention

The invention is further illustrated by the following examples. The example should not be construed as further limiting. The animal models used throughout the Examples are accepted animal models and the demonstration of efficacy in these animal models is predictive of efficacy in humans.

It was determined that tegaserod, a partial 5-HT₄ receptor agonist and inhibitor of 5-HT₂B receptors, applied alone or in combination with a PPI, has a protective effect against mucosal injury and gastric muscle dysfunction induced by indomethacin. Rats were treated with tegaserod (1 mg/kg), omeprazole (20 mg/kg), a combination of both drugs or the vehicle one day prior to the experiment and 30 minutes prior to intragastric infusion of indomethacin (50 mg/kg). Animals were euthanized 6 hours post-indomethacin and gastric ulceration and inflammation were evaluated. Circular muscle strips from the gastric antrum of indomethacin-treated rats receiving vehicle showed increased contractile responses to high KCl, carbachol, 5-HT and electrical field stimulation (EFS, 1-15 Hz) of enteric nerves. Pretreatment with tegaserod and omeprazole applied individually or in combination, reduced gastric ulceration and mucosal inflammation. Indomethacin-induced hypercontractility was unaffected by omeprazole, while it was significantly attenuated by tegaserod or the combined treatment. The results suggest that improvement of gastric muscle contractility by tegaserod offers protection against NSAID-induced gastric damage and may advance the protective effect of omeprazole when both drugs are administered in combination.

### 1. Materials and Methods

### 1.1. Animals

Male Sprague-Dawley rats (Charles River, Laboratories, Wilmington, Mass.) weighing 250-300 g were housed two in a cage at standard conditions (21-23° C., controlled humidity, 12 hour light/dark cycle). The animals were allowed to acclimate for one week prior to initiation of drug treatment. The experimental procedures were approved by the Animal Care and Use Committees at the V.A. Medical Center and the University of Oklahoma Health Science Center, Oklahoma City, Okla.

### 1.2. Indomethacin-Induced Gastric Damage

Prior to the experiment the rats were fasted overnight with free access to water. On the day of experiment the rats received intragastric gavage of 50 mg/kg indomethacin dissolved in 2% NaCO₃ and brought to a volume of 1.5 mL in saline (pH 6.8). Indomethacin was given at 7:30 A.M. and the animals were then placed in their home cage with free access to food and water. The rats were euthanized 6 h post-indomethacin infusion, the stomach was isolated and placed in Ca²⁺-free Krebs buffer aerated with 95% O₂ and 5% CO₂.

### 1.3. Drug Treatment

Tegaserod and omeprazole were dissolved in propylene glycol (100%). Drugs were prepared fresh for each experiment and stored at 4° C. between treatments. Rats were randomly assigned to 3 treatment groups (6 rats per group) receiving 1 mg/kg tegaserod, 20 mg/kg omeprazole or a combination of 1 mg/kg tegaserod and 20 mg/kg omeprazole, respectively. A separate control group received treatment with propylene glycol only. The drugs or the vehicle were administered intraperitoneally at a volume of 0.1 mL/100 g body weight at 8 A.M. and 5 P.M. one day prior to the experiment and on the day of experiment 30 minutes prior to indomethacin.

### 1.4. Evaluation of Mucosal Injury

The stomach was cut along the small curvature, cleansed of ingested food and the fundus was separated from the glandular portion of the stomach. Both regions were pinned flat to the silgard-covered bottom of a dish filled with Ca²⁺-free Krebs buffer continuously aerated with 95% O₂ and 5% CO₂ and placed on the stage of a dissecting microscope. Mucosal ulceration, observed as redness and bleeding along the surface of mucosal folds was pronounced in the glandular stomach but was absent in the fundic area. The total length of hemorrhagic lesions, which were approximately 1 mm wide, was measured and served as an index of ulceration. The mucosa was carefully dissected from the muscle and harvested for measurement of myeloperoxidase (MPO) activity.

### 1.5. Myeloperoxidase Activity

Following dissection, the mucosal tissue was snap-frozen in liquid nitrogen and stored at −80° C. and MPO activity was assayed simultaneously. Homogenization and extraction of MPO from the homogenate by subsequent freeze-thaw sessions were carried out in hexadodecyl-trimethylamonium bromide (HTAB) phosphate buffer (pH 6). MPO activity was tested in 10 μl samples using 3,340,5,5′-tetramethylbenzidine TMB Microwell Peroxidase Substrate System (Sigma, St. Louis, Mo.) and horseradish peroxidase (HRP) as a relative standard. MPO activity was expressed as equivalent to the activity of the relative standard (ng of HRP) converting the same amount of TMB substrate for 10 min at room temperature. The data were expressed in ng normalized per g wet weight of the tissue.

### 1.6. Smooth Muscle Contractility

Krebs buffer free of Ca²⁺ was used during tissue isolation to minimize muscle contractility and avoid overstretching. Circular smooth muscle strips (10 mm×2 mm) were isolated from the gastric antrum and suspended in organ baths under an initial load of 0.5 g (5 mN). The bathing solution was then switched to Krebs containing 2.5 mM Ca²⁺ and isometric tone and spontaneous phasic contractions were recorded after a 90-minute equilibration. Optimal tension was adjusted by loading the preparation and examining the contractile response to 80 mM KCl. The experimental protocols were designed to characterize indomethacin-induced changes in smooth muscle contractility and the ability of tegaserod and omeprazole to prevent smooth muscle dysfunction. Experiments was performed to investigate: 1) contractile responses induced by receptor-independent KCl depolarization; 2) concentration-response curves to carbachol or 5-HT due to activation of smooth muscle muscarinic receptors or 5-HT receptors; 3) electrical field stimulation (EFS) of enteric nerves regulating gastric contractility. A pair of platinum electrodes positioned parallel to the muscle strip and a Grass S88 stimulator (Grass Institute Division, W. Warwick, R.I.) were used to deliver 10-s trains of rectangular pulses with 0.5 ms pulse-duration applied at increasing frequencies of 1, 5, 10 and 15 Hz. Tetrodotoxin (1 μM) was added to the bathing solution to verify that the responses to EFS are neurally mediated. At the end of the experiment the muscle strips were blotted dry and weighed.

### 1.7. Solutions and Drugs

The Krebs buffer was of the following composition (mM): NaCl 120, KCl 6, MgCl₂ 1.2, NaH₂PO₄ 1.2, CaCl₂ 2.5, NaHCO₃ 14.4 and glucose 11.5 (pH 7.3-7.4 when aerated with 95% O₂ and 5% CO₂). Tegaserod maleate was supplied by Novartis Pharmaceuticals (East Hanover, N.J.). Omeprazole, indomethacin, carbamylcholine chloride, atropine sulfate, 5-hydroxytryptamine creatinine sulfate and tetrodotoxin were obtained from Sigma (St. Louis, Mo.) and were dissolved in distilled water. All drugs were added to the baths in volumes less that 1% of the total bath volume.

### 1.8. Data Analysis and Statistics

Contractile responses were measured as changes in resting tension (mN) and were normalized for gram wet weight of tissue. Data are presented as means±SEM from preparations isolated from 5-6 animals in each experimental group. When establishing the models indomethacin-induced abnormalities were evaluated by a comparison to naïve rats using Student t-test. The effects of omeprazole, tegaserod, a combined treatment or the vehicle in indomethacin-treated rats were compared using ordinary one-way ANOVA or non-parametric Kruskal-Willis ANOVA followed by Bonferroni or the non-parametric Dunn's test for multiple comparison. Difference were considered significant at p<0.05.

### 2. Results

### 2.1. Protective Effects Against Indomethacin-Induced Gastric Mucosal Injury

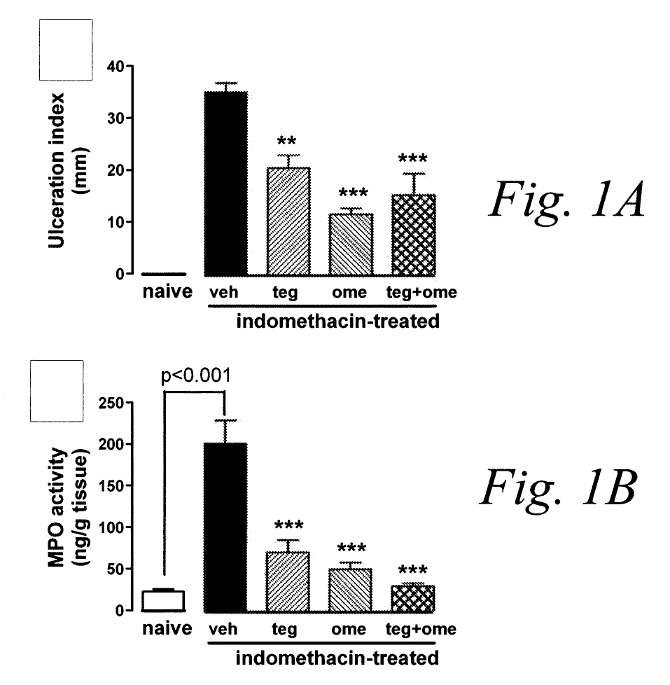

Gastric ulceration was detected along the surface of mucosal folds in the glandular part of the stomach but was absent in the fundus 6 hours after administration of indomethacin. Mean values of the ulceration index are presented in FIG. 1A. Mucosal MPO activity, measured as a marker of neutrophil infiltration, was significantly elevated in the glandular stomach of indomethacin-treated rats compared to naïve rats (FIG. 1B). Pretreatment of the rats with tegaserod, omeprazole or a combination of both drugs significantly reduced the formation of gastric lesions compared to rats pretreated with vehicle only. However, despite the significant reduction in the ulceration index, hemorrhagic lesions were still present in the rats receiving omeprazole and tegaserod treatment. In contrast, pretreatment with omeprazole and tegaserod administered either alone or in combination, inhibited MPO activity to levels that were not significantly different from those in naïve animals. No significant differences were found between the MPO levels obtained in the groups pretreated with omperazole and tegaserod administered alone or in combination.

### 2.2. Protective Effects Against Indomethacin-Induced Gastric Muscle Dysfunction

2.2.1. Hypercontractility in Response to KCl.

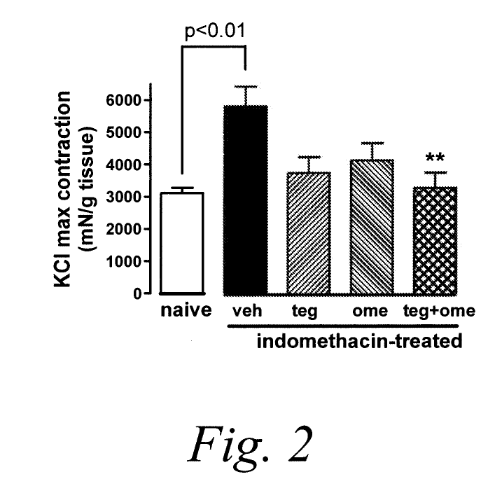

The spontaneous contractile activity of the antral smooth muscles was characterized by low basal tone with phasic contractions developing at a frequency of 4-6 per minute. Exposure to high KCl induced a tonic contraction in all circular muscle strips isolated from the gastric antrum, however, the maximal amplitude of KCl-induced contractions was significantly higher (p<0.01) in the strips isolated from indomethacin-treated rats undergoing pretreatment with the vehicle compared to naïve rats. Tegaserod and omeprazole administered individually showed a tendency to prevent the development of hypercontractile responses to KCl, however in the effect of neither of the drugs reached statistical significance compared to the vehicle. Only when tegaserod and omeprazole were administered in combination, the amplitude of KCl-induced contractions was significantly lower compared to the responses in vehicle-treated animals (FIG. 2).

2.2.2. Hypercontractility in Response to Carbachol.

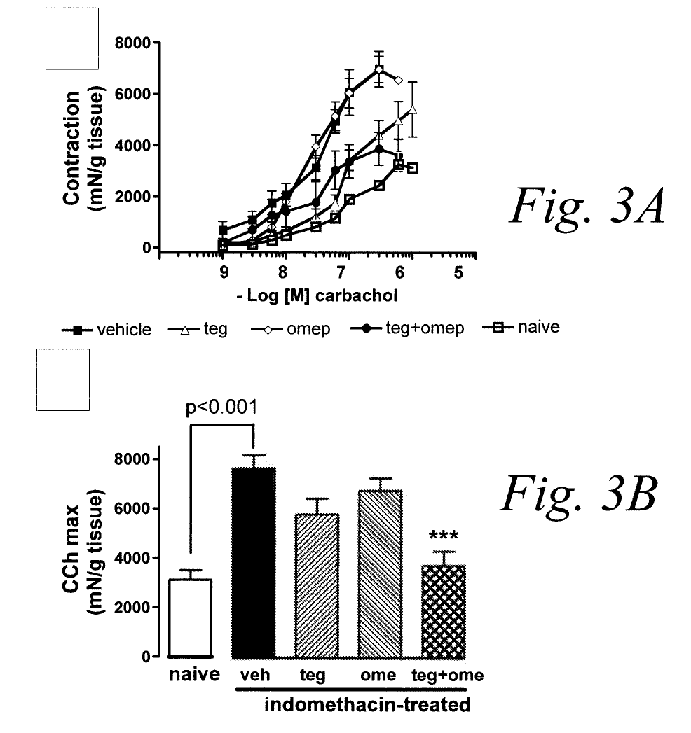

Circular muscle strips isolated from the gastric antrum of both indomethacin-treated and naïve rats responded to increasing concentrations of carbachol (1 nM-10 mM) added to the bathing solution with concentration-dependent contractions. Comparison of the concentration-response curves showed a significant increase in the amplitude of the contractile responses in strips from indomethacin-treated rats receiving vehicle compared to naïve rats (FIG. 3 A). The increased reactivity of the antral muscle appeared without a significant shift in sensitivity to the muscarinic receptor agonist with similar EC₅₀ values in naïve and indomethacin-treated rats (naïve rats: log EC₅₀=6.95, 95% CL-7.38 to −6.51, Hill slope 1.07 vs. vehicle pretreated rats receiving indomethacin: log EC50=−7.32, 95% CL −7.62 to −7.01, Hill slope 1.11). Pretreatment of the rats with a combination of tegaserod and omeprazole resulted in a significant attenuation of the maximal carbachol-induced contraction compared to the effect of the vehicle (FIG. 3B). In comparison, the effects of tegaserod or omeprazole administered individually were not significantly different from the effect the vehicle.

2.2.3. Hypercontractile Responses to 5-HT.

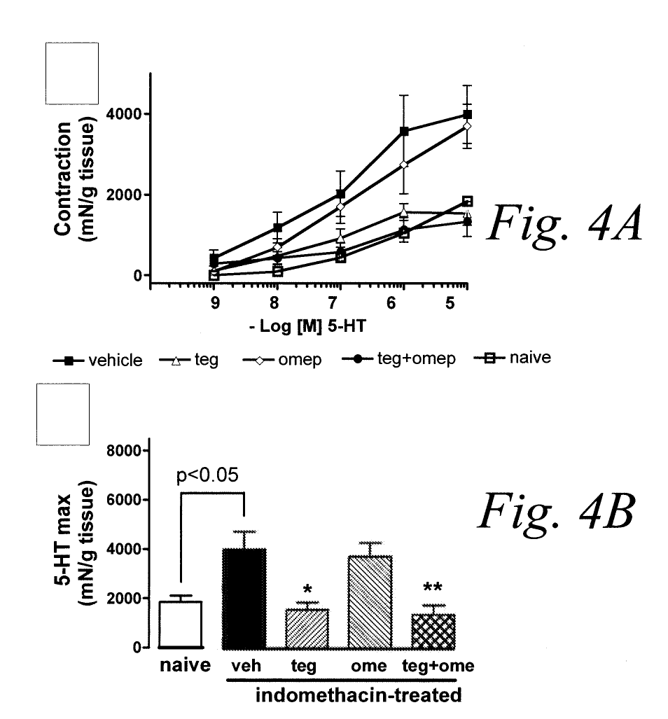

Antral smooth muscle strips from naïve or indomethacin-treated rats responded to administration of 5-HT to the bathing solution with concentration-dependent contractions. The amplitude of contractile responses in muscle strips from naïve rats was significantly (p<0.05) lower compared to the responses in strips from indomethacin-treated rats receiving vehicle. However, there was no significant shift in the concentration response curve to 5-HT (log EC₅₀=6.299, 95% CL-7.097 to −5.776, Hill slope 0.48 in naïve rats vs. log EC₅₀=6.839, 95% CL −8.028 to −5.805, Hill slope 0.65) (FIG. 4A). Pretreatment of the rats with tegaserod alone or a combination of tegaserod and omeprazole caused a significant reduction in the amplitude of the maximal response to 5-HT compared to the effect of the vehicle (FIG. 4B). In comparison, the contractile response in strips from omeprazole pretreated rats did not differ significantly from the response in preparations from vehicle-treated animals.

2.2.4. Hypercontractile Responses to EFS of Enteric Nerves.

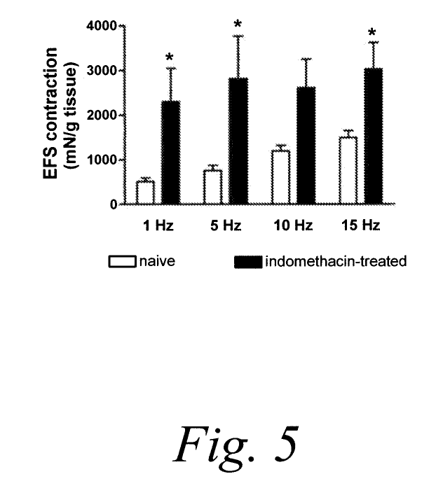

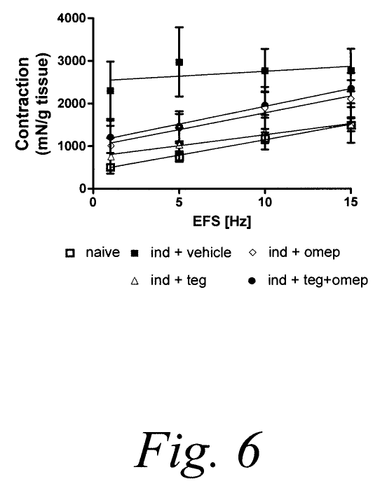

EFS applied at frequency of 1-15 Hz induced frequency-dependent responses characterized by an initial inhibition of spontaneous activity or a low amplitude relaxation followed by a pronounced contraction. Both the inhibitory and the excitatory response were abolished by administration of tetrodotoxin (1 μM) to the bathing solution indicating that EFS-induced responses are neurogenic. In muscles isolated from naïve rats EFS-induced contractile responses increased in proportion to the frequency of stimulation. A comparison between the amplitude of EFS-induced contractions in muscle strips from naïve and indomethacin-treated animals demonstrated a significant increase in indomethacin-treated rats dosed with vehicle (FIG. 5). Moreover, these preparations responded with high-amplitude contractions to low frequency of stimulation and showed no linear increase of the contractile responses with the increase in EFS frequency (Table 1, slope not significantly different from zero). The protective effects of tegaserod and omeprazole against indomethacin-induced hypercontractility in responses to EFS were evaluated comparing the results obtained by linear regression of frequency-responses data (FIG. 6). Pretreatment of the rats with tegaserod or the combination of tegaserod and omeprazole prevented the increase of contractile responses to EFS and preserved the linear frequency-response relationship (Table 1). Omeprazole applied individually preserves the linear frequency-response relationship, but was able to prevent the increase in amplitude of the contractile responses (FIG. 6).

TABLE 1
Effects of tegaserod and omeprazole on the linear frequency-response
relationship of the contractile responses induced by EFS
(0.5 ms, 1-15 Hz) in antral muscle strips

| | | Indomethacin-treated rats | | | |
|---|---|---|---|---|---|
| Regression | | | | | Omeprazole and |
| Analysis | Naïve rats | Vehicle | Tegaserod | Omeprazole | Tegaserod |
| Slope | 72.3 ± 5.1 | 25.5 ± 2.8 | 51.9 ± 5.1 | 78.8 ± 9.5 | 83.7 ± 4.3 |
| R2 | 0.99 | 0.25 | 0.98 | 0.97 | 0.99 |
| F | 345.5 | 0.68 | 102.6 | 67.8 | 379.9 |
| P (deviation | 0.003 | 0.49 (n.s.) | 0.009 | 0.01 | 0.002 |
| from 0) | | | | | |

Regression analysis of data from 5-6 rats per group, slopes are mean ± SEM.

The protective effects of tegaserod, omeprazole or their combination against the development indomethacin-induced gastric abnormalities are summarized in Table 2. The gastric damage induced by acute intragastric administration of indomethacin in the rat was characterized by gastric mucosal ulceration and inflammation in the glandular stomach, hypercontractile responses to high KCl, carbachol, 5-HT and EFS. As seen in the Table 2, the combined treatment with tegaserod and omeprazole reduced indomethacin-induced ulceration and mucosal inflammation and attenuated the development of smooth muscle hypercontractility, while individual treatment with omeprazole was effective against some but not all of the observed abnormalities.

TABLE 2
Ability of tegaserod and omeprazole administered alone or in combination
to prevent the development of gastric mucosal injury and antral
muscle hypercontractility induced by indomethacin

| | Prevention by pretreatment with | | | |
|---|---|---|---|---|
| Abnormalities | | | | Omeprazole |
| induced by | | | | and |
| indomethacin | Vehicle | Tegaserod | Omeprazole | Tegaserod |
| Gastric ulceration | − | + | + | + |
| Inflammation(MPO) | − | + | + | + |
| Hypercontractility | − | + | − | + |
| kcl | | | | |
| Hypercontractility | − | + | − | + |
| carbachol | | | | |
| Hypercontractility | − | + | + | + |
| EFS | | | | |

1. A pharmaceutical composition comprising tegaserod and a proton pump inhibitor.

2. The pharmaceutical composition of claim 1, wherein the proton pump inhibitor is selected from the group consisting of omeprazole, esomeprazole, lansoprazole, rabeprazole, pantoprazole and leminoprazole.

3. The pharmaceutical composition of claim 1, wherein the proton pump inhibitor is omeprazole.

4. A method of treating or preventing gastric injury in a subject in need thereof, comprising administering to the subject an effective amount of tegaserod.

5. The method of claim 4, wherein the gastric injury is associated with NSAID administration.

6. The method of claim 4, wherein the tegaserod is administered in combination with a proton pump inhibitor.

7. The method of claim 6, wherein the proton pump inhibitor is selected from the group consisting of omeprazole, esomeperazole, lansoprazole, rabeprazole, pantoprazole and leminoprazole.

8. The method of claim 6, wherein the proton pump inhibitor is omeprazole.

9. The method of claim 6, wherein tegaserod is administered first followed by administration of a proton pump inhibitor.

10. The method of claim 6, wherein the proton pump inhibitor is administered first followed by administration of tegaserod.

11. The method of claim 6, wherein tegaserod and the proton pump inhibitor are administered simultaneously.

12. The use of claim 4, wherein the gastric injury is selected from the group consisting of mucosal injury, gastric muscle dysfunction, gastritis, peptic erosions, ulcerations and gastric lesions.

13. A method of treating pain and/or inflammation in a subject, said method comprising administering to the subject in need thereof a therapeutically-effective amount of a pharmaceutical composition comprising an NSAID and tegaserod.

14. The method of claim 13, wherein the NSAID is selected from the group consisting of salicylic acid, aspirin, methyl salicylate, diflunisal, salsalate, olsalazine, sulfasalazine, acetaminophen, sulindac, etodolac, tolmetin, ketorolac, diclofenac, ibuprofen, naproxen, fenoprofen, ketoprofen, flurbiprofen, oxaprozin, indomethacin, piroxicam, celecoxib, lumiracoxib, and rofecoxib.

15. The method of claim 13, wherein the inflammation is selected from the group consisting of fever, arthritis, asthma, bronchitis, menstrual cramps, tendinitis, bursitis, inflammatory disorders of the skin, gastrointestinal conditions, irritable bowel syndrome, functional dyspepsia, vascular diseases, migraine headaches, periarteritis nodosa, thyroidiris, aplastic anemia, Hodgkin's disease, sclerodoma, rheumatic fever, myasthenia gravis, sarcoidosis, nephrotic syndrome, Behcet's syndrome, polymyositis, hypersensitivity, conjunctivitis, gingivitis, swelling occurring after injury and myocardial ischemia.

16. The method of claim 15, wherein the arthritis is selected from rheumatoid arthritis, spondyloarthopathies, gouty arthritis, systemic lupus erythematosus, osteoarthritis and juvenile arthritis.

17. The method of claim 13, wherein the pain is selected from the group consisting of menstrual pain, low back pain, neck pain, skeletal pain, post-partum pain, headache, pain associated with migraine, toothache, sprains, strains, arthritis, degenerative joint diseases, gout, ankylosing spondylitis, bursitis, burns, including radiation and corrosive chemical injuries, sunburns, bone fracture, immune and autoimmune diseases, cellular neoplastic transformations or metastic tumor growth, and pain following surgical and dental procedures.

18. A method of treating mucosal injury and/or gastric muscle dysfunction in a subject in need thereof, comprising administering to the subject an effective amount of tegaserod.

19. The method of claim 18, wherein the tegaserod is administered in combination with omeprazole.

20. The method of claim 18, wherein the subject is human.
